SEQUENCE LISTING (18 sequences)

SEQ ID NO: 1 - DNA, 339 bp, Artificial Sequence
  source: /note=“Description of Artificial Sequence Synthetic polynucleotide”
gcagccaccatggtgctccagacccaggtgttcatcagcctgctgctgtggatcagcggcgcctacggcgatatcgacgcctgcgccgacgccgatgccgacgctagcgccgagcagaagctgatcagcgaagaggacctgaacggagccgtggacgaacagaaactgatctccgaggaggatctgaacgccgtcggccaggacacccaggaagtgatcgtcgtcccccacagcctgcccttcaaggtggtggtgatcagcgccatcctggccctggtggtgctgaccatcatctccctgatcatcctgattatgctgtggcagaagaagccccgttga

SEQ ID NO: 2 - protein, 109 aa, Artificial Sequence
  source: /note=“Description of Artificial Sequence Synthetic polypeptide”
MVLQTQVFISLLLWISGAYGDIDACADADADASAEQKLISEEDLNGAVDEQKLISEEDLNAVGQDTQEVIVVPHSLPFKVVVISAILALVVLTIISLIILIMLWQKKPR

SEQ ID NO: 3 - DNA, 703 bp, Artificial Sequence
  source: /note=“Description of Artificial Sequence Synthetic polynucleotide”
cgccaccatggcctgggctctgctgctcctcaccctcctcactcagggcacaggatcctgggctgatatcgaactgacccagccgccttcagtgagcgttgcaccaggtcagaccgcgcgtatctcgtgtagcggcgataatattggtaataagtatgtttcttggtaccagcagaaacccgggcaggcgccagttgttgtgatttatggtgataataatcgtccctcaggcatcccggaacgctttagcggatccaacagcggcaacaccgcgaccctgaccattagcggcactcaggcggaagacgaagcggattattattgctcttcttatgattcttcttattttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 4 - DNA, 1407 bp, Artificial Sequence
  source: /note=“Description of Artificial Sequence Synthetic polynucleotide”
atgaaacacctgtggttcttcctcctgctggtggcagctcccagatgggtcctgtcccaggtgcaattggtggaaagcggcggcggcctggtgcaaccgggcggcagcctgcgtctgagctgcgcggcctccggatttaccttttcttcttatggtatgcattgggtgcgccaagcccctgggaagggtctcgagtgggtgagcaatatctattctgatggtagcaataccttttatgcggatagcgtgaaaggccgttttaccatttcacgtgataattcgaaaaacaccctgtatctgcaaatgaacagcctgcgtgcggaagatacggccgtgtattattgcgcgcgtaatatgtatcgttggccttttcattatttttttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcagggcaacgtgttcagctgtagcgtgatgcacgaggccctgcacaaccactacacccagaagagcctgagcctgtgctaa

SEQ ID NO: 5 - protein, 231 aa, Artificial Sequence
  source: /note=“Description of Artificial Sequence Synthetic polypeptide”
MAWALLLLTLLTQGTGSWADIELTQPPSVSVAPGQTARISCSGDNIGNKYVSWYQQKPGQAPVVVIYGDNNRPSGIPERFSGSNSGNTATLTISGTQAEDEADYYCSSYDSSYFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 468 aa, Artificial Sequence
  source: /note=“Description of Artificial Sequence Synthetic polypeptide”
MKHLWFFLLLVAAPRWVLSQVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLC

SEQ ID NO: 7 - DNA, 1650 bp, Artificial Sequence
  source: /note=“Description of Artificial Sequence Synthetic polynucleotide”
atgaaacacctgtggttcttcctcctgctggtggcagctcccagatgggtcctgtcccaggtgcaattggtggaaagcggcggcggcctggtgcaaccgggcggcagcctgcgtctgagctgcgcggcctccggatttaccttttcttcttatggtatgcattgggtgcgccaagcccctgggaagggtctcgagtgggtgagcaatatctattctgatggtagcaataccttttatgcggatagcgtgaaaggccgttttaccatttcacgtgataattcgaaaaacaccctgtatctgcaaatgaacagcctgcgtgcggaagatacggccgtgtattattgcgcgcgtaatatgtatcgttggccttttcattatttttttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcagggcaacgtgttcagctgtagcgtgatgcacgaggccctgcacaaccactacacccagaagagcctgagcctgtcccccggcaaggccgctgccgagcagaagctgattagcgaagaggacctgaatggggccgtggacgaacagaaactgatctccgaggaggacctgaacgccgtgggccaggacacccaggaagtgatcgtcgtcccccacagcctgcccttcaaggtggtggtgatcagcgccatcctggccctggtggtgctgaccatcatcagcctgatcatcctgattatgctgtggcagaaaaagccccgctga

SEQ ID NO: 8 - protein, 549 aa, Artificial Sequence
  source: /note=“Description of Artificial Sequence Synthetic polypeptide”
MKHLWFFLLLVAAPRWVLSQVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAEQKLISEEDLNGAVDEQKLISEEDLNAVGQDTQEVIVVPHSLPFKVVVISAILALVVLTIISLIILIMLWQKKPR

SEQ ID NO: 9 - protein, 30 aa, Artificial Sequence
  source: /note=“Description of Artificial Sequence Synthetic polypeptide”
MKKLLFAIPLVVPFYSHSDYCDIEFAETVE

SEQ ID NO: 10 - protein, 78 aa, Artificial Sequence
  source: /note=“Description of Artificial Sequence Synthetic polypeptide”
AAAEQKLISEEDLNGAVDEQKLISEEDLNAVGQDTQEVIVVPHSLPFKVVVISAILALVVLTIISLIILIMLWQKKPR

SEQ ID NO: 11 - protein, 16 aa, Homo sapiens
LHNHYTQKSLSLSPGK

SEQ ID NO: 12 - protein, 35 aa, Artificial Sequence
  source: /note=“Description of Artificial Sequence Synthetic polypeptide”
LHNHYTQKSLSLSPGKAAAEQKLISEEDLNGAVDE

SEQ ID NO: 13 - protein, 13 aa, Artificial Sequence
  source: /note=“Description of Artificial Sequence Synthetic peptide”
LHNHYTQKSLSLC

SEQ ID NO: 14 - protein, 470 aa, Artificial Sequence
  source: /note=“Description of Artificial Sequence Synthetic polypeptide”
MKHLWFFLLLVAAPRWVLSQVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPC

SEQ ID NO: 15 - protein, 472 aa, Artificial Sequence
  source: /note=“Description of Artificial Sequence Synthetic polypeptide”
MKHLWFFLLLVAAPRWVLSQVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKC

SEQ ID NO: 16 - protein, 478 aa, Artificial Sequence
  source: /note=“Description of Artificial Sequence Synthetic polypeptide”
MKHLWFFLLLVAAPRWVLSQVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHKHKHKC

SEQ ID NO: 17 - protein, 110 aa, Artificial Sequence
  source: /note=“Description of Artificial Sequence Synthetic polypeptide”
MVLQTQVFISLLLWISGAYGDYCDIEFAETVEASAEQKLISEEDLNGAVDEQKLISEEDLNAVGQDTQEVIVVPHSLPFKVVVISAILALVVLTIISLIILIMLWQKKPR

SEQ ID NO: 18 - protein, 101 aa, Artificial Sequence
  source: /note=“Description of Artificial Sequence Synthetic polypeptide”
MVLQTQVFISLLLWISGAYGDICASAEQKLISEEDLNGAVDEQKLISEEDLNAVGQDTQEVIVVPHSLPFKVVVISAILALVVLTIISLIILIMLWQKKPR

(19) United States
(12) Patent Application Publication
(10) Pub. No.: US 2010/0317546 A1
(43) Pub. Date: Dec. 16, 2010

(54) Display Vectors and Methods and Uses Thereof

(75) Inventors: Markus Enzelberger, Planegg (DE); Philippe Thiel, Planegg (DE); Solveig Peters, Munich (DE); Rüdiger Neun, Munich (DE); Stephen Yoder, Freising-Weihenstephan (DE)

Correspondence Address: MorphoSys AG, Lena-Christ-Str., 48, Martinsried/Planegg, 82152, DE

(73) Assignee: MORPHOSYS AG, Martinsried/Planegg (DE)

(21) Appl. No.: 12/812,236

(22) PCT Filed: Jan. 9, 2009
(86) PCT No.: PCT/EP2009/050237

§ 371 Date: Jul. 27, 2010

Related U.S. Application Data
(60) Provisional application No. 61/020,456, filed on Jan. 11, 2008.

(51) Int. Cl. C40B 40/02; C12N 15/79; C12N 15/85; C12N 5/10; C12N 5/24; C12Q 1/68; C12P 21/00

(52) U.S. Cl. 506/14; 435/320.1; 435/325; 435/358; 435/370; 435/346; 435/6; 435/69.1

(57) ABSTRACT

The present invention relates, in one aspect, to a vector comprising (a) a first polynucleotide capable of encoding a first (poly)peptide comprising at least one cysteine residue, and (b) a second polynucleotide capable of encoding a second (poly)peptide which is a cell surface anchor comprising at least one cysteine residue, wherein the vector is operable in a eukaryotic host cell to express and to cause or allow the attachment of said first (poly)peptide to said second (poly)peptide by formation of a disulfide bond between said cysteine residues comprised within said first (polypeptide and said second (poly)peptide, respectively, wherein said first (poly)peptide is exhibited at the surface of a eukaryotic host cell.

### BACKGROUND OF THE INVENTION

Display technologies are well established in prokaryotic systems. Most prominent are variants of the classical phage display technology (Smith, 1985, Science 228, 1315-1317), but various other technologies exist, such as ribosome display. In contrast, respective technologies in eukaryotic systems suffer from various technological pitfalls and hurdles.

In the present invention we provide novel methods and compositions which, for the first time, enable the efficient display of (poly)peptides on the surface of host cells, such as eukaryotic host cells. The (poly)peptides so displayed are characterized by a reactive cysteine residue which forms a disulfide bond with one or more components of the host cell, which may be a eukaryotic host cell. Such component of the host cell may be another, second, (poly)peptide which is a cell surface anchor. This system allows the efficient display of (poly)peptides on the surface of cells, such as eukaryotic cells, and does not require the generation of fusion polypeptides.

### BRIEF DESCRIPTION OF THE FIGURES

FIG. 1: shows an example of a display vector of the invention.

FIG. 2: shows an example of a display vector of the invention.

FIG. 3: shows the transmembrane domain of PDGFR and the vicinity of the constructs used in the present invention. The constructs comprised tandem myc epitopes as well as a V kappa leader sequence N-terminal to the transmembrane domain of PDGFR. On the top, for comparison, a typical construct for prokaryotic CysDisplay is shown (see for example WO 01/05950 and PCT/EP2008/060931).

FIG. 4: shows the vector map of the vector encoding the polypeptide comprising a PDGFR transmembrane domain and a reactive cysteine residue.

FIG. 5: shows a schematic representation of some of the immunoglobulins used in the study. The original version of MOR3080 (MOR03080) is shown on the top. The sketch in the middle shows the fusion protein in which the heavy chain was fused to the transmembrane domain of the PDGF receptor. This IgG-PDGFRTM fusion polypeptide served as a control. The sketch on the bottom shows a derivative of MOR3080, in which a cysteine residue was introduced at the C-terminus of the heavy chain (various such constructs were generated in the present study, see Example 5). This polypeptide forms a disulfide bridge with the polypeptide comprising the transmembrane domain of PDGFR, into which a cysteine residue was introduced in the N-terminus (also for this part various constructs were generated in the present study, see Example 6).

FIG. 6: shows a schematic representation of the vicinity of the reactive cysteine residue of the immunoglobulins used in the study. The reactive cysteine residue is indicated by the arrow in the sequence shown on the bottom. The sequence in the middle is from the corresponding immunoglobulin-PDGFRTM fusion protein, which does not comprise a reactive cysteine residue, just as the original MOR3080, which is shown on the top.

FIG. 7: shows a vector map of a representative vector encoding the immunoglobulin-PDGFRTM fusion protein used in the present invention. The heavy chain is derived from MOR3080, an immunoglobulin specific for CD38.

FIG. 8: shows a vector map of a representative vector encoding the immunoglobulin used in the present invention, into which a cysteine residue was introduced at the C-terminus of the heavy chain. The heavy chain is derived from MOR3080, an immunoglobulin specific for CD38.

FIG. 9: shows the expression of the various constructs of the present invention as measured by FACS analysis. Row 1 shows results with cells which have not been transfected (mock transfection). Row 2 shows results with cells which have been transfected with the construct Cys-PDGFRTM. Row 3 shows results with cells which have been transfected with the construct Cys-IgG. Row 4 shows results with cells which have been transfected with the constructs Cys-IgG and Cys-PDGFRTM, combined as double transfection. Row 5 shows the results of the cells transfected with the IgG-PDGFRTM fusion construct. Detection in column A was performed with an anti-myc antibody and in column B with an anti IgG antibody. In column C biotinylated antigen was used and its detection was performed with a labeled streptavidin. All stainings were done separately. myc-containing cell surface proteins were detected when PDGFRTM was expressed (alone, with Cys-IgG or as part of the fusion protein). Likewise, IgG was detected when IgG was expressed. Transfection of Cys-IgG led to a significant surface expression and ligand binding activity. When Cys-IgG was co-transfected with Cys-PDGFRTM, an increase in IgG staining as well as in CD38-binding could be seen (dashed lines).

FIG. 10: shows the amino acid sequences of the four Cys-IgG variants tested in the present study.

FIG. 11: shows the expression of the four Cys-IgG variants tested in the present studies. Detected were IgG (column A) and antigen (column B), respectively. Rows 1-5 show the flow cytometric results of non-transfected (mock transfected) cells (row 1) and of cells transfected with Cys-IgG constructs A-D, respectively (rows 2-5). Analysed was the surface expression of IgG, and of antigen-binding activity (CD38-binding activity). MOR3080-derived immunoglobulins comprising a reactive cysteine residue (Cys-IgG) were transfected into Flp-In CHO cells. Expression was monitored by flow cytometric analysis of IgG (anti-IgG). Binding of MOR03080 ligand was analyzed by adding biotinylated CD38 into the culture medium and detecting bound ligand with streptavidin. All stainings were done separately. Transfection of all four Cys-IgG variants led to a significant surface expression and ligand binding activity.

FIG. 12: shows the amino acid sequences of the three Cys-PDGFRTM variants tested in the present study.

FIG. 13: shows the result of the analysis of the rebinding of secreted IgG. Analysed was the surface expression of IgG, and the intracellular expression of EGFP. MOR3080-derived immunoglobulin comprising a reactive cysteine residue (Cys-IgG) was transfected into CHO-K1 cells. Other CHO-K1 cells were transfected with EGFP. Expression was monitored by flow cytometric analysis of IgG (anti-IgG) and of EGFP. Low staining of IgG could be observed for the EGFP-expressing cells after co-culturing of both kinds of cells. Column A shows the preparations without antibody staining, cell surface expression of Cys-IgG is depicted in column B. Non-transfected cells expressing EGFP are shown in row 1, parental cells transfected with Cys-IgG are shown in row 2. Row 3 shows CHO cells stably transfected to intracellularly express EGFP which have been co-cultured with cells that have been transiently transfected with the Cys-IgG variant construct A.

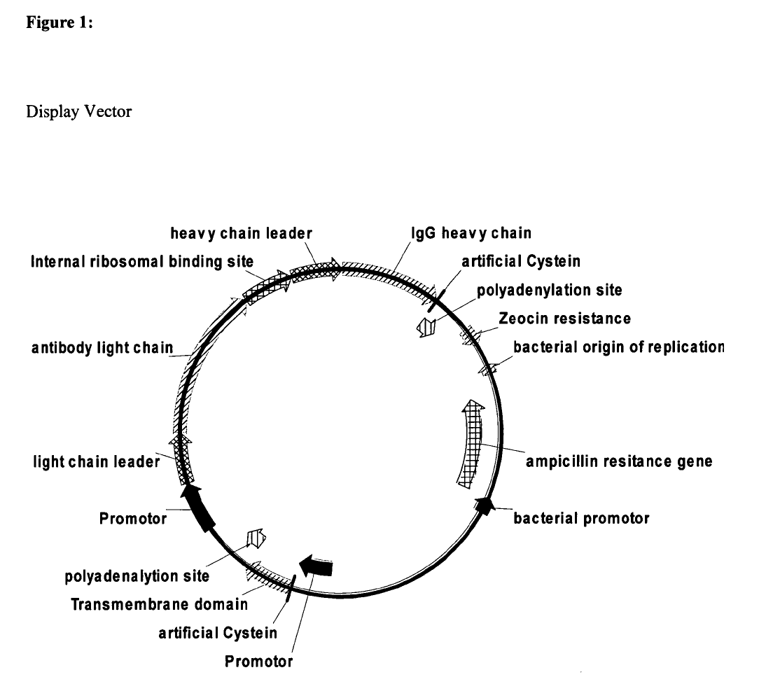

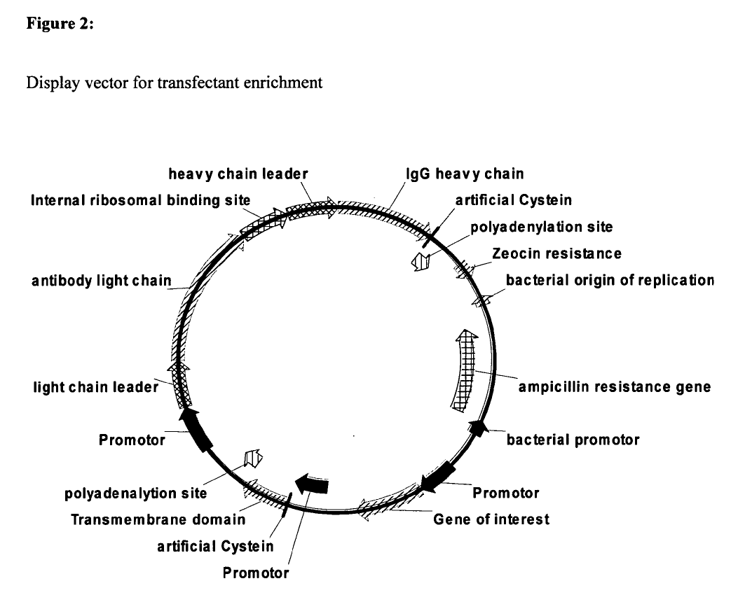

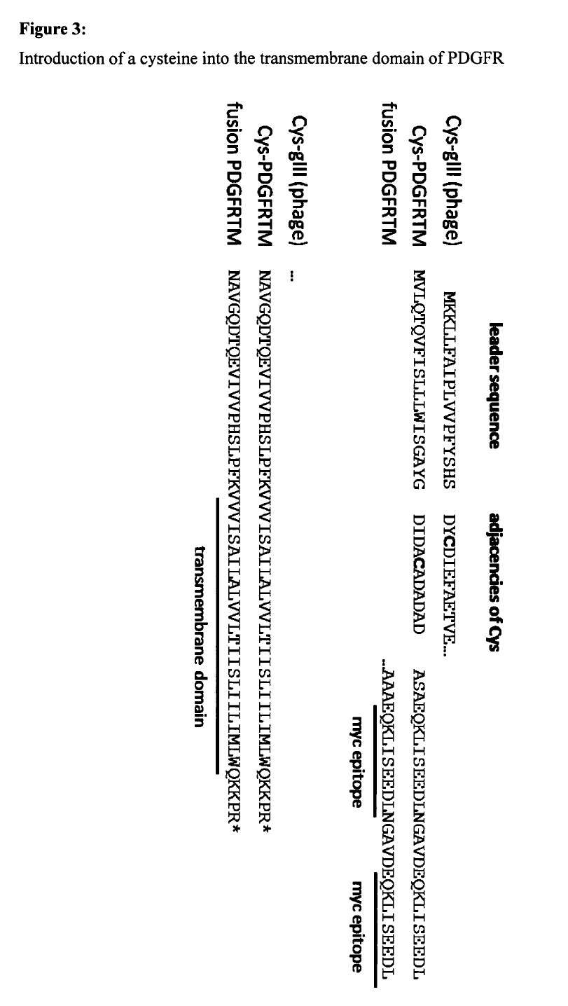

### DESCRIPTION OF THE INVENTION

The present invention relates, in one aspect, to a vector comprising (a) a first polynucleotide capable of encoding a first (poly)peptide comprising at least one cysteine residue, and (b) a second polynucleotide capable of encoding a second (poly)peptide which is a cell surface anchor comprising at least one cysteine residue, wherein the vector is operable in a host cell, which may be a eukaryotic host cell, to express and to cause or allow the attachment of said first (poly)peptide to said second (poly)peptide by formation of a disulfide bond between said cysteine residues comprised within said first (poly)peptide and said second (poly)peptide, respectively, wherein said first (poly)peptide is exhibited at the surface of a host cell. In another aspect, the present invention relates to a vector comprising a first polynucleotide capable of encoding a first (poly)peptide comprising at least one cysteine residue, wherein the vector is operable in a host cell, which may be a eukaryotic host cell, to express and to cause or allow the attachment of said first (poly)peptide to said host cell by formation of a disulfide bond between said cysteine residues comprised within said first (poly)peptide and a component of the host cell, wherein said first (poly)peptide is exhibited at the surface of the host cell.

In another aspect the present invention relates to a vector comprising (a) a first polynucleotide encoding a first (poly)peptide comprising at least one cysteine residue, and (b) a second polynucleotide encoding a second (poly)peptide which is a cell surface anchor comprising at least one cysteine residue, wherein the vector is operable in a eukaryotic host cell to express and to cause or allow the attachment of said first (poly)peptide to said second (poly)peptide by formation of a disulfide bond between said cysteine residues comprised within said first (poly)peptide and said second (poly)peptide, respectively, wherein said first (poly)peptide is exhibited at the surface of a eukaryotic host cell. In another aspect, the present invention relates to a vector comprising a first polynucleotide encoding a first (poly)peptide comprising at least one cysteine residue, wherein the vector is operable in a host cell to express and to cause or allow the attachment of said first (poly)peptide to said host cell by formation of a disulfide bond between said cysteine residues comprised within said first (poly)peptide and a component of the host cell, wherein said first (poly)peptide is exhibited at the surface of a host cell. In another aspect, the present invention relates to a vector comprising a first polynucleotide encoding a first (poly)peptide comprising at least one cysteine residue, wherein the vector is operable in a eukaryotic host cell to express and to cause or allow the attachment of said first (poly)peptide to said eukaryotic host cell by formation of a disulfide bond between said cysteine residues comprised within said first (poly)peptide and a component of the eukaryotic host cell, wherein said first (poly)peptide is exhibited at the surface of a eukaryotic host cell.

In a preferred embodiment the vector of the present invention, further comprises a signal sequence operably linked to the first polynucleotide.

In another preferred embodiment the vector of the invention further comprises a signal sequence operably linked to the second polynucleotide.

The term “vector” as used in connection with the present invention refers to any vector operable in the host cell, which preferably is a eukaryotic host cell. Said vector can comprise genetic elements needed for the vector to exert its function in a host cell (e.g., a eukaryotic cell), such as, e.g. promoters, restriction sites for endonuclease digests, genes for selection, internal ribosomal entry sites. The skilled artisan is aware of said essential genetic elements defining a eukaryotic vector. The vectors of the present invention further comprise genetic elements comprising cysteine residues.

The term “operable” is to be construed homologous to the term “functional” in connection with the present invention. Thus, a vector “operable in” a host cell (e.g., a eukaryotic cell) is a vector that displays its functions based on host cell-specific genetic elements comprised in said vector, respectively.

The term “(poly)peptide” in the present invention is to be considered in its broadest sense as appreciated by the skilled artisan. Hence, the term “(poly)peptide” as used herein describes a group of molecules which comprises the group of peptides, as well as the group of polypeptides. The group of peptides is consisting of molecules with up to 30 amino acids, the group of polypeptides or proteins is consisting of molecules with more than 30 amino acids. (Poly)peptides of particular interest in connection with the present invention are binding members, as outlined further below.

The terms “cell surface anchor”, “anchor”, “anchor (poly)peptide” refer, inter alia, to any molecular structure connected to or attached to the surface of a eukaryotic cell. Said term comprises structures known to the skilled artisan but also structures being capable of anchorage to the surface not yet known.

In other words, the terms “cell surface anchor”, “anchor”, “anchor (poly)peptide” refer to a (poly)peptide moiety that, on expression in a host cell, becomes attached or otherwise associated with the outer surface of the host cell. An anchor (poly)peptide can be a transmembrane protein moiety, or can be a (poly)peptide moiety otherwise linked to the cell surface (e. g., via post-translational modification, such as by a phosphatidyl-inositol or disulfide bridge). The term encompasses native proteins to the host cell, or exogenous proteins introduced for the purpose of anchoring to the cell surface.

Anchors include any synthetic modification or truncation of a naturally occurring anchor that still retains the ability to be attached to the surface of a host cell or phage particle.

Preferred anchor protein moieties are contained in, for example, cell surface proteins of a eukaryotic cell. Effective anchors include portions of a cell surface protein sufficient to provide a surface anchor when fused to another (poly)peptide, such as a chain of a multi-chain (poly)peptide in accordance with this invention. The use of protein pairs that are separately encoded and expressed but associate at the surface of a cell by covalent (e. g., disulfide) or non-covalent bonds is also contemplated as a suitable anchor.

In another more preferred embodiment of the present invention, the cell surface anchor is selected from the group consisting of: a-agglutinin, the a-agglutinin component Agalp and Aga2p, FLO1, PDGF, PRIMA, mDAF, and other natural occurring or synthetic membrane anchor molecules known to the skilled artisan. In certain preferred embodiments, the cell surface anchor is PDGF, or a derivative or fragment thereof. In yet other preferred embodiments, the cell surface anchor comprises the transmembrane domain of PDGF (hereinafter called “PDGFTM”).

The term “exhibited” at or on the surface of a cell, such as a eukaryotic cell, is equivalent to the term “displayed” at or on the surface of a cell, such as a eukaryotic cell. The polypeptide so exhibited or displayed is functional to be used in the vectors, methods and uses of the present invention. In particular, a polypeptide so exhibited or displayed is able to interact with other polypeptides via a reactive cysteine residue.

The term “surface” in the term “surface of a eukaryotic host cell” refers to any structure surrounding the cellular body of any of the known eukaryotic host cells. The skilled artisan is aware of such structures, including, for example, a plasma membrane, but also a cellular wall of, e.g., a plant or a fungus cell. The term “plasma membrane” in connection with the present invention is to be construed as comprising any eukaryotic membrane as understood by the skilled artisan to be comprised in said term. Therefore, said term also comprises, e.g., structures such as the Endoplasmic Reticulum or the Golgi vesicles inside eukaryotic cells.

The term “at least one cysteine residue” is to be understood in connection with the present invention that the (poly)peptide may contain exactly 1 single cysteine residue, but also at least 2, at least 3, 5, at least 5, 10, at least 10, at least 20, 50, at least 50, at least 100 or even more cysteine residues.

The term “express” in the context “wherein the vector is operable in a [eukaryotic] host cell to express and to cause or allow the attachment of said first (poly)peptide to said second (poly)peptide by formation of a disulfide bond . . . ”, or in a similar context, refers to the situation that the vector comprises genetic elements capable of driving the transcription of, e.g. a polynucleotide encoding a (poly)peptide. Said elements are well known to the skilled artisan and comprise, e.g. eukaryotic promoters and polyadenylation signals. In particular, it is to be understood that the (poly)peptide is expressed in a eukaryotic host cell prior to the attachment of the (poly)peptide to the cell surface. The expression of polynucleotides encoding said (poly)peptide and the step of causing or allowing the attachment may be performed in separated steps and/or environments. Preferably, however, expression and the step of causing or allowing the attachment take place sequentially in an appropriate host cell.

The term “signal sequence” or “leader sequence” is well known to the skilled artisan and refers to any sequence which enables to target a (poly)peptide expressed from a polynucleotide comprising said signal sequence to a specific location in the cell. A preferred cellular location in connection with the present invention is the plasma membrane that forms the cell surface of the eukaryotic cell. The signal sequence, as understood for the present invention, may be part of the first and/or second polynucleotide.

Once the vector is transfected into the host cell, such as a eukaryotic host cell, expression of the first and/or second (poly)peptide can be either constitutive or inducible. The resulting (poly)peptides can be linked via the formation of a disulfide bond thus joining together the first and second (poly)peptide. The first (poly)peptide can thus be presented on the cell surface of the host cell via its linkage to the second (poly)peptide, i.e. the cell surface anchor. Alternatively, the first (poly)peptides can be linked via the formation of a disulfide bond with a component of the host cell, such as a eukaryotic host cell. The first (poly)peptide can thus be presented on the cell surface of the host cell, such as a eukaryotic host cell.

Host Cells

In a preferred embodiment, said first (poly)peptide is a single-chain (poly)peptide, a term well known by the skilled artisan. In the context of the present invention, single-chain (poly)peptides having the capacity of being functional as binding members, as also outlined further below, are preferred. Most preferred single chain (poly)peptides are scFvs.

In another preferred embodiment, the first (poly)peptide of the vector of the present invention comprises a first chain of a binding molecule multi-chain (poly)peptide. It is more preferred that the vector of the present invention further comprises: (a1) a third polynucleotide capable of encoding a third (poly)peptide, wherein said third (poly)peptide comprises a second chain of the binding molecule multi-chain (poly)peptide. It is also preferred that the vector of the present invention further comprises:

(a2) a fourth polynucleotide capable of encoding a fourth (poly)peptide, wherein said fourth (poly)peptide comprises a third chain of the binding molecule multi-chain (poly)peptide.

In another preferred embodiment, the vector of the present invention, further comprises:

(a3) a fifth polynucleotide capable of encoding a fifth (poly)peptide, wherein said fifth (poly)peptide comprises a fourth chain of the binding molecule multi-chain (poly)peptide.

In a further preferred embodiment, the first, second, third, fourth and/or fifth polynucleotide of the vector of the present invention are functionally linked.

The term “functionally linked” or “operably linked” as used in connection with the present invention refers to the situation that any of the above polynucleotides not necessarily needs to be present on the same vector as long as any functional connection, such as coordinated expression, exists between the chains. Additionally, the vectors of the invention also may comprise IRES sequences instead of promoters for linking expression and translation of two polynucleotides. Any correlation the skilled artisan is aware of in the above context may be envisaged.

In yet a further preferred embodiment of the vector of the present invention, the multi-chain (poly)peptide comprises a two-chain (poly)peptide.

In a preferred embodiment of the vector of the present invention, the multi-chain (poly)peptide comprises a four-chain (poly)peptide, wherein the four-chain (poly)peptide is comprised of two first chains and two second chains.

The above particularly preferred embodiments of the present invention refer to vectors wherein the (poly)peptide which is exhibited or displayed on the surface of a host cell, such as a eukaryotic host cell, is a multi-chain (poly)peptide. Said term in connection with the present invention refers to a functional (poly)peptide comprised of two or more discrete (poly)peptide elements (i.e. “chains”) covalently or non-covalently linked together by molecular association other than by peptide bonding.

The chains of a multi-chain (poly)peptide can be the same or different. A prominent example of a multi-chain (poly)peptide is an immunoglobulin (e. g., IgA, IgD, IgE, IgG, and IgM), typically composed of four chains, two heavy chains and two light chains, which assemble into a multi-chain (poly)peptide in which the chains are linked via several disulfide (covalent) bonds. Active immunoglobulin Fab fragments, involving a combination of a light chain (LC) domain and a heavy chain (HC) domain, form a particularly important class of multi-chain (poly)peptides. As well as forming a disulfide bond, the light chain and heavy of a Fab are also known to effectively associate (non-covalently) in the absence of any disulfide bridge. Other examples of multi-chain (poly)peptides include, but are not limited to, the extracellular domains of T cell receptor (TCR) molecules, MHC class I molecules and MHC class II molecules.

Preferably, the multi-chain (poly)peptide encoded by vector (s) of the present invention exists as either a two-, three-, or multi-chain (poly)peptide. More preferably, the multi-chain (poly)peptide is a two-chain or four-chain (poly)peptide comprised of two different chains. More preferably, the multi-chain (poly)peptide is selected from a group of multi-chain (poly)peptides consisting of T cell receptors, MHC class I molecules, MHC class II molecules, and immunoglobulin Fab fragments. More preferably, the multi-chain (poly)peptide is an IgA, IgD, IgE, IgG, IgM, or biologically active fragment thereof. Also more preferably, the multi-chain (poly)peptide is a Fab.

The multi-chain (poly)peptide may refer to any multi-chain peptide known to the skilled artisan. Preferred in the context of the present invention are binding molecules. Said binding molecules are capable of forming complexes with specific targets, when brought into contact. Preferred binding molecules are immunoglobulins and Fabs. The immunoglobulin may be a full length immunoglobulin in which a cysteine residue was added. Such additional cysteine residue may be added at any of the termini of the immunoglobulin chains, such as the N-terminus of the immunoglobulin heavy chain, the C-terminus of the immunoglobulin heavy chain, N-terminus of the immunoglobulin light chain or the C-terminus of the immunoglobulin light chain. In particular embodiments additional cysteine residue is added at the C-terminus of the heavy chain. Such additional cysteine residue may be also added near any of the termini of the immunoglobulin chains (e.g. the C-terminus of the heavy chain), e.g. within two, three, five, ten, twenty, fifty, one hundred, two hundred, three hundred or five hundred amino acids of the termini of a immunoglobulin chain.

The immunoglobulin may also be a variant of an immunoglobulin which retains the binding properties of the native immunoglobulin. For example, the immunoglobulin may lack the last, the two last, the three last, the four last, the five last, at least the five last, at least the ten last, at least the twenty last, at least the fifty last or at least the one hundred last amino acids at the C-terminus or the N-terminus. In yet other examples, the immunoglobulin may comprise additional amino acids, such as at least one, at least two, at least three, at least five, at least ten, at least twenty, at least fifty or at least one hundred additional amino acids at the C-terminus or the N-terminus. In yet other examples an amino acid of a native immunoglobulin is substituted to a cysteine residue.

In yet other embodiments the cysteine residues involved in the formation of the disulfide bond that leads to the exhibition of the (poly)peptide on the cell surface of the host cell is located within a peptide stretch, or is located adjacent to other amino acid residues which positively affect the reactivity of the cysteine residue. The term “positively affects the reactivity” as used in the context of the present invention refers to a situation where the equilibrium of a reaction in which two thiol groups react to form a disulphide bond, is shifted towards the side of the product, i.e. a higher number of disulphide bonds is formed. In certain embodiments one of the two cysteines residues which form said disulfide bond is located within a positively charged peptide stretch, or is located adjacent to positively charged amino acids. In other embodiments one of the two cysteines residues which form said disulfide bond is located within a negatively charged peptide stretch, or is located adjacent to negatively charged amino acids. In yet other embodiments one of the two cysteines residues which form said disulfide bond is located within a positively charged peptide stretch or is located adjacent to positively charged amino acids, and the other of the two cysteines residues which form said disulfide bond is located within a negatively charged peptide stretch or is located adjacent to negatively charged amino acids. Said positively or negatively charged amino acids may be directly adjacent, within two amino acids, within three amino acids, within five amino acids, within ten amino acids, within twenty amino acids, or within two amino acids next to said cysteine residue, provided they positively affect the reactivity of said cysteine residue. In certain preferred embodiments there are more than one, more than, two, more than three, more than five or more than ten charged amino acids (positively or negatively charged, respectively) involved in the generation of a charged environment which positively affects the reactivity of said cysteine residue. Preferred positively charge amino acids are histidine, lysine and arginine. Most preferred positively charge amino acids are lysine and arginine. Preferred negatively charge amino acids are aspartic acid and glutamic acid.

In yet another preferred embodiment of a vector of the present invention, the first, and/or second, and/or third, and/or fourth chain is/are attached via said disulfide bond(s) to the (poly)peptide which is a cell surface anchor. In yet another preferred embodiment of the vector of the present invention, the first, and/or second, and/or third, and/or fourth chain is/are attached via said disulfide bond(s) to a component of the host cell.

The (poly)peptide and/or multi-chain (poly)peptide in accordance with the present invention, may be attached via one or several disulfide bonds to the cell surface anchor or to a component of the host cell. For any (poly)peptide comprising more than one single chain as outlined above for the multi-chain (poly)peptides, said attachment may occur by forming a disulfide bond between either chain and the cell surface anchor (i.e., e.g., the first chain and the anchor molecule or the second chain and the anchor molecule), or by forming a disulfide bond between either chain and a component of the host cell (i.e., e.g., the first chain and a component of the host cell or the second chain and a component of the host cell). Attachment of the anchor molecule or the component of the host cell to more than one of the (poly)peptide chains may also occur.

In a preferred embodiment of the vector of the present invention, the anchor comprises a cell surface protein of a eukaryotic cell. Said cell surface proteins are well known to the skilled artisan.

In a more preferred embodiment of the vector of the present invention, the anchor comprises a portion of a cell surface protein of a eukaryotic cell that anchors to the cell surface of the eukaryotic host cell.

In another more preferred embodiment of the vector of the present invention, the anchor is selected from the group consisting of: a-agglutinin, the a-agglutinin component Agalp and Aga2p, FLO1, PDGF, PRIMA, mDAF, and other natural occurring or synthetic membrane anchor molecules known to the skilled artisan.

The term a “component of the host cell” refers to any native or endogenous component of the host cell of the present invention (in contrast to the cell surface anchors of the present invention, which are typically artificial or exogenous molecules). Such components of the host cell act as a reaction partner of the [first] (poly)peptide which comprises at least one cysteine residue to form a disulfide bond. Said formation of the disulfide bond leads to the exhibition of the [first] (poly)peptide at the surface of the host cell. Typically, the components of the host cell to be used in the present invention are molecules of exterior compartments of the host cells, such as components of the cell wall, the cell membrane, the inner membrane, the outer membrane, the periplasm, or components attached to any of the above.

In yet another preferred embodiment of the vector of the present invention, either said at least one cysteine residue comprised in said first (poly)peptide or said at least one cysteine residue comprised in said second (poly)peptide has been introduced artificially. In a more preferred embodiment, said at least one cysteine residue comprised in said first (poly)peptide has been introduced artificially. In another more preferred embodiment, said at least one cysteine residue comprised in said second (poly)peptide has been introduced artificially. In a most preferred embodiment of the vector of the present invention said at least one cysteine residue comprised in said first (poly)peptide and said at least one cysteine residue comprised in said second (poly)peptide have been introduced artificially.

The above particularly preferred embodiments relate to the situation that one or more cysteine residue(s) have been introduced artificially into, e.g., the cell surface anchor and/or into, e.g., the (poly)peptide which will be displayed on the surface of the host cell. In this context, the term “artificially introduced” is to be understood likewise as the term “non-naturally occurring”. It refers to a situation that a wild type or naturally occurring (poly)peptide has been modified by, e.g., recombinant means. For example, a nucleic acid encoding a naturally occurring PDGFR transmembrane domain may be manipulated by standard procedures to introduce a cysteine codon creating a nucleic acid sequence encoding a modified domain, wherein a cysteine residue is artificially introduced by insertion into, or addition of said cysteine residue to, said domain, or by substitution of an amino acid residue comprised in said domain by said cysteine residue, or by any combination of said insertions, additions, or substitutions. Any other method known to the skilled artisan in the above context is also considered in the scope of the present invention. Upon expression from the vector of the present invention of the polynucleotide comprising such, e.g., recombinantly introduced cysteine codon, a mutant (poly)peptide is formed comprising a cysteine residue.

In a preferred embodiment, the vector is integrated into the genome. The skilled artisan is aware of eukaryotic vector integration systems. Said systems may also be used in connection with the vectors of the present invention.

Any prokaryotic or eukaryotic cell may be used as a host cell in the present invention. Preferred host cells are eukaryotic host cells. More preferred host cells are mammalian host cells. Even more preferred host cells are primate host cells. Most preferred host cells are human host cells. A eukaryotic host cell as contemplated in connection with the present invention, refers to any eukaryotic cell known to the skilled artisan. Therefore, said term comprises, inter alia, animal cells, yeast, fungi and plant cells. Exemplary eukaryotic cells include HEK293 cells (ATCC number: CRL-1573), HKB11 cells (Bayer Schering Pharma), and CHO cells. The polypeptides produced by the eukaryotic cells of the present invention contain post-tranlational modifications, such as glycosylation patterns, of the respective eukaryotic host cells.

The present invention also relates to a composition comprising: (a) a first vector comprising a first polynucleotide capable of encoding a first (poly)peptide comprising at least one cysteine residue, wherein said first (poly)peptide comprises a first chain of a binding molecule multi-chain (poly)peptide; and (b) a second vector comprising a second polynucleotide capable of encoding a second (poly)peptide comprising at least one cysteine residue which is a cell surface anchor; and, optionally, (c) a third vector comprising a third polynucleotide capable of encoding a third (poly)peptide, wherein said third (poly)peptide comprises a second chain of the binding molecule multi-chain (poly)peptide; and, optionally, (d) a fourth vector comprising a fourth polynucleotide capable of encoding a fourth (poly)peptide, wherein said fourth (poly)peptide comprises a third chain of the binding molecule multi-chain (poly)peptide, and, optionally, (e) a fifth vector comprising a fifth polynucleotide capable of encoding a fifth (poly)peptide, wherein said fifth (poly)peptide comprises a fourth chain of the binding molecule multi-chain (poly)peptide, wherein the vectors are operable in a eukaryotic host cell to express and to cause or allow the attachment of said first (poly)peptide to said second (poly)peptide by formation of a disulfide bond between said cysteine residue comprised within said first (poly)peptide and said cysteine residue comprised within said second (poly)peptide, wherein said first and, optionally, said third, fourth, fifth (poly)peptide is exhibited at the surface of a eukaryotic host cell.

The present invention also relates to a composition comprising: (a) a first vector comprising a first polynucleotide capable of encoding a first (poly)peptide comprising at least one cysteine residue, wherein said first (poly)peptide comprises a first chain of a binding molecule multi-chain (poly)peptide; and, optionally, (b) a second vector comprising a second polynucleotide capable of encoding a second (poly)peptide, wherein said second (poly)peptide comprises a second chain of the binding molecule multi-chain (poly)peptide; and, optionally, (c) a third vector comprising a third polynucleotide capable of encoding a third (poly)peptide, wherein said third (poly)peptide comprises a third chain of the binding molecule multi-chain (poly)peptide, and, optionally, (d) a fourth vector comprising a fourth polynucleotide capable of encoding a fourth (poly)peptide, wherein said fourth (poly)peptide comprises a fourth chain of the binding molecule multi-chain (poly)peptide, wherein the vectors are operable in a host cell, such as a eukaryotic host cell, to express and to cause or allow the attachment of said first (poly)peptide to a component of the host cell by formation of a disulfide bond between said cysteine residue comprised within said first (poly)peptide and said component of the host cell, wherein said first and, optionally, said second, third, and fourth (poly)peptide is exhibited at the surface of the host cell, such as a eukaryotic host cell.

The above embodiment of the present invention is to be construed such that any of the polynucleotides of the invention as defined further above, i.e. the first, second, third, fourth or fifth polynucleotide, may be comprised in 1, 2, 3, 4 or 5 or even more vectors. Any permutations deriving thereof and conceivable for the skilled artisan are comprised in the scope of the present invention. The skilled artisan knows that the polynucleotide capable of coding for the cell surface anchor and the polynucletide(s) coding for the single chain or multi-chain (poly)peptide which will be exhibited on the surface of the cell are to be functionally linked, as already outlined further above. Hence, combinations of vectors or vector sets are also envisaged in the scope of the present invention.

In another embodiment, the present invention relates to a host cell comprising a vector of the present invention or the composition of the present invention. In a preferred embodiment, the host cell of the present invention is a eukaryotic host cell. In more preferred embodiment the eukaryotic host cell of the present invention is a mammalian host cell. In even more preferred embodiment the mammalian host cell of the present invention is a primate host cell. In most preferred embodiment the primate host cell of the present invention is a human host cell.

The present invention also relates to a vector library comprising a plurality of vectors of the present invention, wherein said plurality is derived from a heterogeneous population of the first, and/or third, and/or fourth and/or fifth (poly)peptide.

It is further envisaged, in connection with the above embodiment, that more than one cell surface anchor may be utilized. Hence, also the second (poly)peptide, i.e. the cell surface anchor, may be heterogeneous.

Additionally, the present invention relates to a display library, such as a eukaryotic display library, comprising a population of cells, such as eukaryotic cells, collectively displaying a heterogeneous population of at least 10² (poly)peptides as defined in the present invention's vector(s). Preferably, display libraries, such as eukaryotic display libraries, comprising at least 10³, 10⁴, 10⁵, more preferably at least 10⁶ or at least 10⁷ (poly)peptides are envisaged in the scope of the present invention.

Furthermore, the present invention relates to a host cell library, such as a eukaryotic host cell library, obtainable by transfecting the vector library of the present invention into a plurality of host cells.

The skilled artisan is aware of how to construct a library as contemplated in the above embodiments.

The present invention also relates to a method for displaying a (poly)peptide as defined in the present invention's vector(s) on the surface of a host cell comprising the steps of: (a) introducing into a host cell at least one vector of the present invention or the composition of the present invention; and (b) culturing the host cells under conditions suitable for expression of the (poly)peptides comprised in said vector(s) or said composition. Preferably said host cell is a eukaryotic host cell.

As outlined above and in other terms, of particular interest in the present invention is the display of a (poly)peptide of interest on the surface of a eukaryotic cell, preferably a mammalian cell. Of particular advantage in this respect is the situation that the (poly)peptide to be displayed is linked via one or more disulfide bond(s) to the cell surface anchor. Said bond may be cleaved under mild reducing conditions, therefore opening up, for the skilled artisan, new and surprising fields of applications as also shown further below.

As also outlined above, and of likewise interest in the present invention is the display of a (poly)peptide of interest on the surface of a host cell, preferably a eukaryotic cell, more preferably a mammalian cell. Of alternative particular advantage in this respect is the situation that the (poly)peptide to be displayed is linked via one or more disulfide bond(s) to a component of the host cell. Said bond may be cleaved under mild reducing conditions, therefore opening up, for the skilled artisan, new and surprising fields of applications as also shown further below.

In a preferred embodiment, the host cell in the method of the present invention, or in the library of the present invention is a mammalian cell.

Additionally, the present invention relates to a method comprising the steps of: (a) transfecting a population of eukaryotic host cells with at least one vector as defined in the present invention or with the composition of the present invention or the vector library of the present invention, such that substantially each cell comprises a vector or composition encoding a diverse binding member; (b) culturing the host cells under conditions suitable for expression and display on the cell surface of the binding member comprised in said vector or said composition, wherein the attachment of the binding member to the (poly)peptide which is a cell surface anchor is achieved by formation of a disulfide bond; (c) allowing for binding of at least one binding member displayed on the cell surface to its target, thereby allowing for the formation of a specific binding member-target complex; and (d) eluting under reducing conditions the cells displaying the at least one specific binding member of step (c). In a preferred embodiment, the above method further comprises the additional step of (c1) carrying out after step (c): washing of the cells which have not bound specifically to the target.

Additionally, the present invention relates to a method comprising the steps of: (a) transfecting a population of host cells, such as eukaryotic host cells, with at least one vector as defined in the present invention or with the composition of the present invention or the vector library of the present invention, such that substantially each cell comprises a vector or composition encoding a diverse binding member; (b) culturing the host cells under conditions suitable for expression and display on the cell surface of the binding member comprised in said vector or said composition, wherein the attachment of the binding member to the component of the host cell is achieved by formation of a disulfide bond; (c) allowing for binding of at least one binding member displayed on the cell surface to its target, thereby allowing for the formation of a specific binding member-target complex; and (d) eluting under reducing conditions the cells displaying the at least one specific binding member of step (c). In a preferred embodiment, the above method further comprises the additional step of (c1) carrying out after step (c): washing of the cells which have not bound specifically to the target.

As used in connection with the above method and also in connection with the invention, the term “binding member” is used in a synonymous way to the term “binding molecule” or “binding moiety”. Said terms, in connection with the present invention, are construed to comprise, inter alia, any scaffold known to a skilled artisan. A “scaffold” in connection with the present invention refers to any collection of (poly)peptides having a common framework and at least one variable region. Scaffolds known to the skilled artisan are, for example, fibronectin based scaffolds or ankyrin repeat protein based scaffolds.

As shown, e.g., in the Examples hereinbelow, the above method allows for the specific elution of binder molecules. Hence, specific (or when using negative selection: non-specific, i.e. non-binding) library members can be isolated.

In another preferred embodiment the above method further comprises the step of determining the nucleic acid sequence of the specific binding member. The identified binding molecule may then be used for further applications known to the skilled artisan. The identified molecule can, for example, be expressed in soluble or conjugated form.

Furthermore, the present invention, in another aspect, relates to a method comprising the steps of: (a) transfecting a population of eukaryotic host cells with at least one vector as defined in the present invention or the composition of the present invention, wherein said vector or said composition comprise(s) a polynucleotide capable of encoding a (poly)peptide comprising a binding member capable of binding to a target; and a gene of interest functionally linked to the (poly)peptide which is a cell surface anchor and/or functionally linked to said binding member; (b) culturing the host cells under conditions suitable for expression and display on the cell surface of the binding member comprised in said vector, wherein the attachment of the binding member to the (poly)peptide which is a cell surface anchor is achieved by formation of a disulfide bond; (c) allowing for binding of the binding member displayed on the cell surface to its target, thereby allowing for the formation of a specific binding member-target complex; and (d) eluting under reducing conditions the cells displaying the specific binding member of step (c). It is more preferred that the above method further comprises the additional step of (c1) carrying out after step (c): washing of the cells which have not bound specifically to the target.

Furthermore, the present invention, in another aspect, relates to a method comprising the steps of: (a) transfecting a population of host cells, such as eukaryotic host cells, with at least one vector as defined in the present invention or the composition of the present invention, wherein said vector or said composition comprise(s) a polynucleotide capable of encoding a (poly)peptide comprising a binding member capable of binding to a target; and a gene of interest functionally linked to said binding member; (b) culturing the host cells under conditions suitable for expression and display on the cell surface of the binding member comprised in said vector, wherein the attachment of the binding member to a component of the host cell is achieved by formation of a disulfide bond; (c) allowing for binding of the binding member displayed on the cell surface to its target, thereby allowing for the formation of a specific binding member-target complex; and (d) eluting under reducing conditions the cells displaying the specific binding member of step (c). It is more preferred that the above method further comprises the additional step of (c1) carrying out after step (c): washing of the cells which have not bound specifically to the target.

Most preferred, said gene of interest is selected from the group consisting of therapeutic proteins, industrial enzymes, and proteins used in research.

In another preferred embodiment of the method of the present invention, the host cell is a eukaryotic cell, a mammalian cell, a primate cell or a human cell.

In a further aspect, the present invention relates to the use of the vector(s) and/or the composition of the present invention for constructing a library as outlined further above.

The present invention furthermore provides compositions comprising a host cell and a (poly)peptide comprising at least one cysteine residue, wherein said (poly)peptide is exhibited at the surface of said host cell. Preferably, said host cell comprises a nucleic acid molecule encoding said (poly)peptide.

In certain embodiments said host cell is a eukaryotic host cell. In other embodiment said host cell is a mammalian host cell, a primate host cell or a human host cell. In alternative embodiments said host cell is a prokaryotic host cell, such as a bacterial host cell.

In certain aspects of the present invention the cysteine residue comprised in said (poly)peptide forms a disulfide bond with a component of the host cell. In preferred embodiments said component of the host cell is an endogenous component of the host cell, a component of the wild type of the host cell, a naturally occurring component of the host cell or a component which was not artificially introduced into the host cell. In preferred embodiments said (poly)peptide comprising at least one cysteine residue is a (poly)peptide exogenous to the host cell, a (poly)peptide not naturally occurring in the host cell or a (poly)peptide artificially introduced into the host cell. In certain aspects said (poly)peptide comprising at least one cysteine residue is a binding member. In preferred aspects said binding member is an immunoglobulin.

In certain aspect the present invention provides a library comprising a plurality of compositions comprising a host cell and a (poly)peptide comprising at least one cysteine residue, wherein said (poly)peptide is exhibited at the surface of said host cell and wherein at least two of the (poly)peptides comprised in said composition are different. In other aspect at least 5, 10, 100, 1000 or 10000 of the (poly)peptides comprised in said composition are different. In other aspects said (poly)peptides are binding members, such as immunoglobulins. In certain aspects at least one of said binding members comprised in the library is bound to its target, thereby forming a specific binding member-target complex.

In certain aspects the present invention provides an assembly comprising a library, wherein at least one of said binding members comprised in said library is bound to its target, and a device to separate the at least one binding member which is bound to said target from binding members which are not bound to said target. In certain aspects said device is a flow cytometer, such as a FACS machine. Respective devices are known to the person skilled in the art and commercially available (e.g. from BD Biosciences, San Jose, Calif.).

In certain aspects the present invention provides a method to isolate a binding member which is bound to its target from a library according to the present invention, said method comprising the steps of:

(a) subjecting said library to conditions alolwing the isolation of the cell comprising the nucleic acids molecules encoding said binding member, and
(b) recovering said nucleic acid molecule.

The following examples are provided to illustrate the present invention and are not to be construed to be limiting thereof.

### EXAMPLES

### Example 1

### Library Selection

In a eukaryotic expression vector, e.g. a pcDNA vector, comprising polynucleotides coding for a membrane anchor protein with a cysteine residue, a respective signal sequence and a polyadenylation site, as well as antibiotic resistances, a library of binding moieties is inserted such that a cysteine is introduced. The obtained vector is transfected into HEK293 cells under conditions that the binding moiety and the membrane anchor plasmid are expressed.

By forming a disulfide bond the membrane anchor and the binding molecule may be linked, and the binding moiety is presented on the surface of the cell with the genetic information contained in the cell.

The population of cells displaying the different library members is brought into contact with a matrix or surface, e.g. sepharose, presenting the targets against which the cell population is selected. Cells displaying library members binding to the target molecule stick to the matrix or surface, whereas non-binding members are depleted by washing. The cells binding to the target molecule are subsequently eluted by reducing the disulfide bond connecting the binding moiety to the membrane anchor by mild conditions (e.g. 0.01 nM DDT). Afterwards the genetic information encoding the binder specificity is recovered by RT-PCR.

### Example 2

### Enrichment of Cells Transfected with a Gene of Interest

A eukaryotic expression vector comprising a membrane anchor protein with a cysteine residue, a respective signal sequence and a polyadenylation site, as well as antibiotic resistances, a high affinity binding molecule with a cysteine specific against a target (e.g. a hapten (e.g. fluorescein), a peptide (e.g. myc) or a protein)—and the gene of interest to be transfected—is transfected into eukaryotic cells under conditions that the binding molecule and the membrane anchor plasmid are expressed.

Cells containing the gene of interest also display the membrane anchor and the specific binding molecule which is presented via a disulfide bond on the surface of the cell and therefore can be used as marker for cell transfection.

The mix of transfected and non-transfected cells is brought in contact with a solid support, e.g. sepharose, bearing the target the binding molecule is directed against. Transfected cells displaying the binding molecules bind to the support. Non-displaying (i.e. non-transfected cells) are washed away and the transfected cells are recovered by reducing the disulfide bond connecting the binding molecule to the membrane anchor by mild conditions (e.g. 0.01 nM DDT).

### Example 3

### Cloning of the Constructs for the Proof-Of-Concept Experiments

The cell surface membrane anchor protein used in the proof-of concept experiments comprises the transmembrane domain of the human platelet-derived growth factor receptor B (PDGFRB; NP_{—}002600.1). Similar fusion proteins have been used by others for other purposes (Cheng and Roffler 2008, Medicinal Research Reviews, Vol. 28(6), pages 885-928; see also the vector pHook-1 from Invitrogen, (Carlsbad, Calif.)).

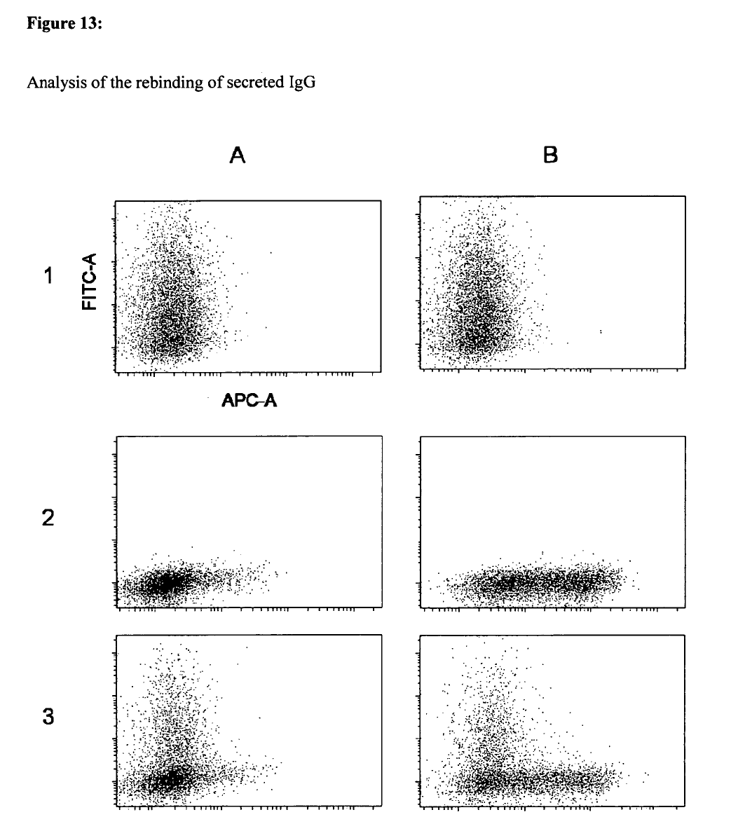

Three types of constructs were generated: (a) Cys-PDGFRTM: a polypeptide comprising the transmembrane domain of PDGFRB and a reactive cysteine residue (various versions of this construct were generated; the construct described in this Example is identical to Cys-PDGFRTM_construct-A of Example 6 and FIG. 13), (b) Cys-IgG: an immunoglobulin of the IgG1-type comprising a reactive cysteine residue at the C-termini of the heavy chains, and (c) IgG-PDGFRTM: a fusion protein comprising an immunoglobulin of the IgGl-type and the transmembrane domain of PDGFRB. The latter served as a control construct.

### Cloning of Cys-PDGFRTM

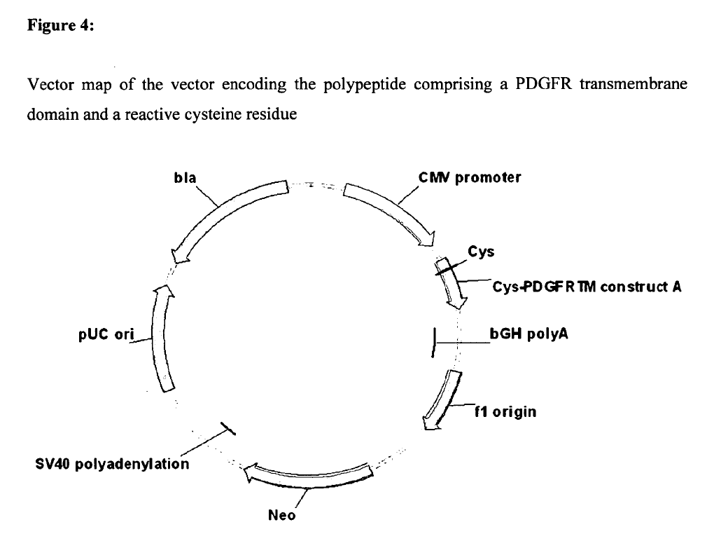

The Cys-PDGFRTM construct comprises (a) the leader sequence of the variable domain for the kappa chain of an immunoglobulin (V kappa) at the N-terminus, followed by (b) a short peptide containing the reactive cysteine residue, followed by (c) a tandem myc epitope, and (d) the transmembrane domain of the human platelet-derived growth factor receptor B (amino acids 512-561 of PDGFRB; NP_{—}002600.1). The short peptide stretch containing the reactive cysteine residues (part (b) of the construct described above) may be an acidic hydrophilic peptide stretch. The nucleic acid sequence encoding this construct was synthesized using optimized codons for expression and contained additional flanking nucleotides encoding restriction sites for subsequent cloning and a Kozak sequence for initiation of translation. The construct was cloned into a standard expression vector (pcDNA3.1) using standard molecular biology techniques. A vector map of the final construct is shown in FIG. 4.

The nucleic acid sequence encoding Cys-PDGFRTM is as follows (the Kozak sequence is underlined):

GCAGCCACCATGGTGCTCCAGACCCAGGTGTTCATCAGCCTGCTGCTGTG
GATCAGCGGCGCCTACGGCGATATCGACGCCTGCGCCGACGCCGATGCCG
ACGCTAGCGCCGAGCAGAAGCTGATCAGCGAAGAGGACCTGAACGGAGCC
GTGGACGAACAGAAACTGATCTCCGAGGAGGATCTGAACGCCGTCGGCCA
GGACACCCAGGAAGTGATCGTCGTCCCCCACAGCCTGCCCTTCAAGGTGG
TGGTGATCAGCGCCATCCTGGCCCTGGTGGTGCTGACCATCATCTCCCTG
ATCATCCTGATTATGCTGTGGCAGAAGAAGCCCCGTTGA

The amino acid sequence of Cys-PDGFRTM is as follows (the leader sequence is underlined; the peptide comprising the reactive cysteine residue is shown in italics; the tandem myc epitope is shown in bold; the transmembrane domain is double underlined):

MVLQTQVFISLLLWISGAYGDIDACADADADASAEQKLISEEDLNGAVDE
QKLISEEDLNAVGQDTQEVIVVPHSLPFKVVVISAILALVVLTIISLIIL
IMLWQKKPR

### Cloning of Cys-IgG

The immunoglobulin used in the proof-of concept experiment, herein referred to as Cys-IgG, is based on MOR3080, an anti-CD38 antibody (see WO 05/103083). It comprises two light chains and to heavy chains, each with leader sequence. A cysteine residue was introduced at the C-terminus of the heavy chain. The light chain was not changed.

Nucleic acids were obtained by standard PCR technology using oligonucleotides encoding additional restriction sites for cloning, a Kozak sequence for the expression cassette (beginning with the light chain) and the codon for the cysteine residue in the heavy chain. The nucleic acid sequence encoding the IgG-PDGFRTM fusion protein (see below) was used as a template. The inserts were cloned into a standard expression vector containing the CMV promoter and an IRES element to allow for cassette expression of both chains controlled by one promoter.

The nucleic acid sequence encoding the light chain of Cys-IgG is as follows (this is also the light chain for IgG-PDGFRTM; the Kozak sequence is underlined):

CGCCACCATGGCCTGGGCTCTGCTGCTCCTCACCCTCCTCACTCAGGGCA
CAGGATCCTGGGCTGATATCGAACTGACCCAGCCGCCTTCAGTGAGCGTT
GCACCAGGTCAGACCGCGCGTATCTCGTGTAGCGGCGATAATATTGGTAA
TAAGTATGTTTCTTGGTACCAGCAGAAACCCGGGCAGGCGCCAGTTGTTG
TGATTTATGGTGATAATAATCGTCCCTCAGGCATCCCGGAACGCTTTAGC
GGATCCAACAGCGGCAACACCGCGACCCTGACCATTAGCGGCACTCAGGC
GGAAGACGAAGCGGATTATTATTGCTCTTCTTATGATTCTTCTTATTTTG
TGTTTGGCGGCGGCACGAAGTTAACCGTCCTAGGTCAGCCCAAGGCTGCC
CCCTCGGTCACTCTGTTCCCGCCCTCCTCTGAGGAGCTTCAAGCCAACAA
GGCCACACTGGTGTGTCTCATAAGTGACTTCTACCCGGGAGCCGTGACAG
TGGCCTGGAAGGCAGATAGCAGCCCCGTCAAGGCGGGAGTGGAGACCACC
ACACCCTCCAAACAAAGCAACAACAAGTACGCGGCCAGCAGCTATCTGAG
CCTGACGCCTGAGCAGTGGAAGTCCCACAGAAGCTACAGCTGCCAGGTCA
CGCATGAAGGGAGCACCGTGGAGAAGACAGTGGCCCCTACAGAATGTTCA
TAG

The nucleic acid sequence encoding the heavy chain of Cys-IgG is as follows (the cysteine codon is underlined and shown in italics):

ATGAAACACCTGTGGTTCTTCCTCCTGCTGGTGGCAGCTCCCAGATGGGT
CCTGTCCCAGGTGCAATTGGTGGAAAGCGGCGGCGGCCTGGTGCAACCGG
GCGGCAGCCTGCGTCTGAGCTGCGCGGCCTCCGGATTTACCTTTTCTTCT
TATGGTATGCATTGGGTGCGCCAAGCCCCTGGGAAGGGTCTCGAGTGGGT
GAGCAATATCTATTCTGATGGTAGCAATACCTTTTATGCGGATAGCGTGA
AAGGCCGTTTTACCATTTCACGTGATAATTCGAAAAACACCCTGTATCTG
CAAATGAACAGCCTGCGTGCGGAAGATACGGCCGTGTATTATTGCGCGCG
TAATATGTATCGTTGGCCTTTTCATTATTTTTTTGATTATTGGGGCCAAG
GCACCCTGGTGACGGTTAGCTCAGCCTCCACCAAGGGTCCATCGGTCTTC
CCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTT
GGGCACCCAGACCTACATCTGCAACGTGAATCACAAGCCCAGCAACACCA
AGGTGGACAAGAGAGTTGAGCCCAAATCTTGTGACAAAACTCACACATGC
CCACCGTGCCCAGCACCTGAACTCCTGGGGGGACCGTCAGTCTTCCTCTT
CCCCCCAAAACCCAAGGACACCCTCATGATCTCCCGGACCCCTGAGGTCA
CATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGA
GGAGCAGTACAACAGCACGTACCGGGTGGTCAGCGTCCTCACCGTCCTGC
ACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCC
CCGAGAACCACAGGTGTACACCCTGCCCCCATCCCGGGAGGAGATGACCA
AGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGAC
CACGCCTCCCGTGCTGGACTCCGACGGCTCCTTCTTCCTCTACAGCAAGC
TCACCGTGGACAAGAGCAGGTGGCAGCAGGGCAACGTGTTCAGCTGTAGC
GTGATGCACGAGGCCCTGCACAACCACTACACCCAGAAGAGCCTGAGCCT
GTGCTAA

The amino acid sequence of the light chain of Cys-IgG is as follows (this is also the light chain for IgG-PDGFRTM):

MAWALLLLTLLTQGTGSWADIELTQPPSVSVAPGQTARISCSGDNIGNKY
VSWYQQKPGQAPVVVIYGDNNRPSGIPERFSGSNSGNTATLTISGTQAED
EADYYCSSYDSSYFVFGGGTKLTVLGQPKAAPSVTLFPPSSEELQANKAT
LVCLISDFYPGAVTVAWKADSSPVKAGVETTTPSKQSNNKYAASSYLSLT
PEQWKSHRSYSCQVTHEGSTVEKTVAPTECS

The amino acid sequence of the heavy chain of Cys-IgG is as follows:

MKHLWFFLLLVAAPRWVLSQVQLVESGGGLVQPGGSLRLSCAASGFTFSS
YGMHWVRQAPGKGLEWVSNIYSDGSNTFYADSVKGRFTISRDNSKNTLYL
QMNSLRAEDTAVYYCARNMYRWPFHYFFDYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLC

### Fusion Polypeptide IgG-PDGFRTM

The fusion protein IgG-PDGFRTM has the same IgG1 light chain as the construct Cys-IgG. In this construct however the IgG1 heavy chain is fused in-frame to PDGFRTM. This construct serves as a control.

The nucleic acid sequence encoding the heavy chain of IgG-PDGFRTM is as follows:

ATGAAACACCTGTGGTTCTTCCTCCTGCTGGTGGCAGCTCCCAGATGGGT
CCTGTCCCAGGTGCAATTGGTGGAAAGCGGCGGCGGCCTGGTGCAACCGG
GCGGCAGCCTGCGTCTGAGCTGCGCGGCCTCCGGATTTACCTTTTCTTCT
TATGGTATGCATTGGGTGCGCCAAGCCCCTGGGAAGGGTCTCGAGTGGGT
GAGCAATATCTATTCTGATGGTAGCAATACCTTTTATGCGGATAGCGTGA
AAGGCCGTTTTACCATTTCACGTGATAATTCGAAAAACACCCTGTATCTG
CAAATGAACAGCCTGCGTGCGGAAGATACGGCCGTGTATTATTGCGCGCG
TAATATGTATCGTTGGCCTTTTCATTATTTTTTTGATTATTGGGGCCAAG
GCACCCTGGTGACGGTTAGCTCAGCCTCCACCAAGGGTCCATCGGTCTTC
CCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTT
GGGCACCCAGACCTACATCTGCAACGTGAATCACAAGCCCAGCAACACCA
AGGTGGACAAGAGAGTTGAGCCCAAATCTTGTGACAAAACTCACACATGC
CCACCGTGCCCAGCACCTGAACTCCTGGGGGGACCGTCAGTCTTCCTCTT
CCCCCCAAAACCCAAGGACACCCTCATGATCTCCCGGACCCCTGAGGTCA
CATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGA
GGAGCAGTACAACAGCACGTACCGGGTGGTCAGCGTCCTCACCGTCCTGC
ACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCC
CCGAGAACCACAGGTGTACACCCTGCCCCCATCCCGGGAGGAGATGACCA
AGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGAC
CACGCCTCCCGTGCTGGACTCCGACGGCTCCTTCTTCCTCTACAGCAAGC
TCACCGTGGACAAGAGCAGGTGGCAGCAGGGCAACGTGTTCAGCTGTAGC
GTGATGCACGAGGCCCTGCACAACCACTACACCCAGAAGAGCCTGAGCCT
GTCCCCCGGCAAGGCCGCTGCCGAGCAGAAGCTGATTAGCGAAGAGGACC
TGAATGGGGCCGTGGACGAACAGAAACTGATCTCCGAGGAGGACCTGAAC
GCCGTGGGCCAGGACACCCAGGAAGTGATCGTCGTCCCCCACAGCCTGCC
CTTCAAGGTGGTGGTGATCAGCGCCATCCTGGCCCTGGTGGTGCTGACCA
TCATCAGCCTGATCATCCTGATTATGCTGTGGCAGAAAAAGCCCCGCTGA

The amino acid sequence of the heavy chain of IgG-PDGFRTM is as follows (the IgG part is underlined):

MKHLWFFLLLVAAPRWVLSQVQLVESGGGLVQPGGSLRLSCAASGFTFSS
YGMHWVRQAPGKGLEWVSNIYSDGSNTFYADSVKGRFTISRDNSKNTLYL
QMNSLRAEDTAVYYCARNMYRWPFHYFFDYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGKAAAEQKLISEEDLNGAVDEQKLISEEDLN
AVGQDTQEVIVVPHSLPFKVVVISAILALVVLTIISLIILIMLWQKKPR

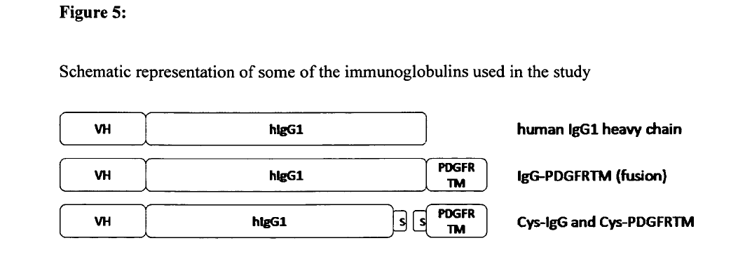

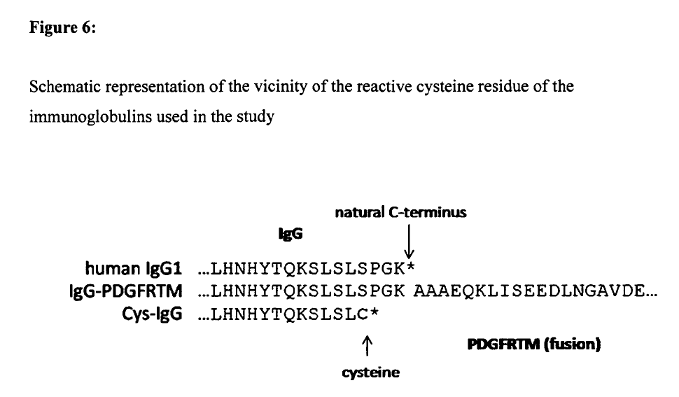

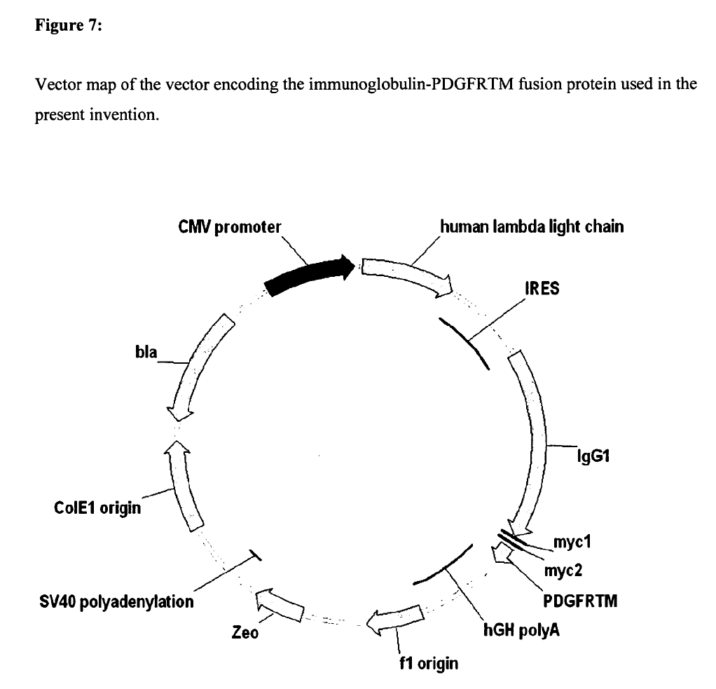

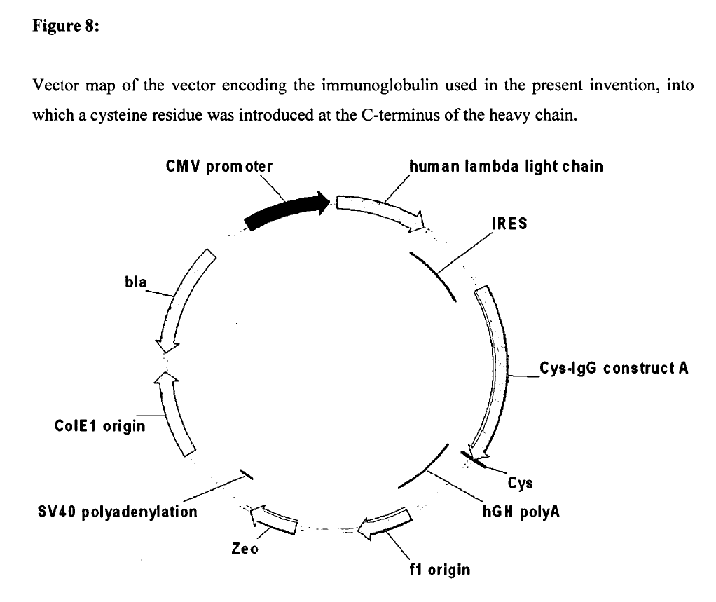

FIG. 5 shows a schematic representation of some of the polypeptides used in the present study. FIG. 6 shows the vicinity of the reactive cysteine residue of the immunoglobulins used in the study. FIG. 7 shows a vector map of the vector encoding the immunoglobulin-PDGFRTM fusion protein used in the present invention. FIG. 8 shows a vector map of the vector encoding the immunoglobulin used in the present invention, into which a cysteine residue was introduced at the C-terminus of the heavy chain.

### Example 4

### Expression of the IgG on the Cell Surface

Flp-In CHO cells (Invitrogen, (Carlsbad, Calif.)) were transiently transfected with the various constructs of the present invention (see Example 3). Expression was analysed by flow cytometric analysis (FACS). Cell culture, transfection, immunofluorescent staining and flow cytometric analysis were performed by standard techniques known in the art.

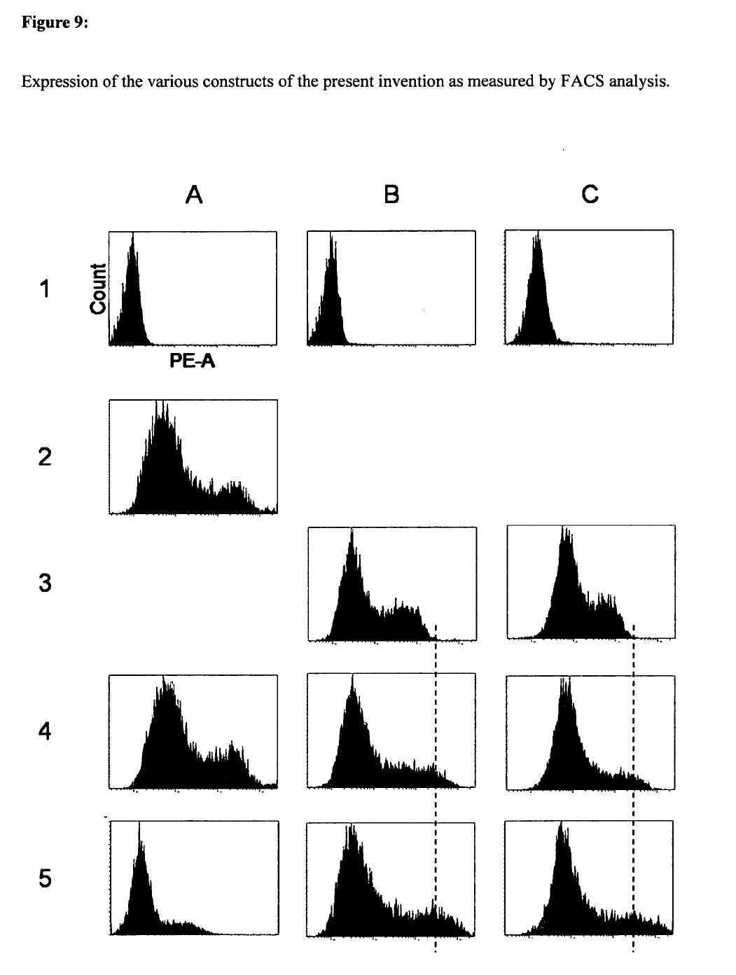

Results are shown in FIG. 9. Row 1 shows results with cells which have not been transfected (mock transfection). Row 2 shows results with cells which have been transfected with the construct Cys-PDGFRTM. Row 3 shows results with cells which have been transfected with the construct Cys-IgG. Row 4 shows results with cells which have been transfected with the constructs Cys-IgG and Cys-PDGFRTM, combined as double transfection. Row 5 shows the results of the cells transfected with the IgG-PDGFRTM fusion construct. Detection in column A was performed with an anti-myc antibody and in column B with an anti IgG antibody. In column C biotinylated antigen was used and its detection was performed with a labeled streptavidin.

Already in cells transfected with Cys-IgG alone (i.e. without co-transfection with a reactive Cys-PDGFRTM counterpart), a substantial anti-IgG signal could be detected. These cells were also able to bind to the CD38 antigen (see FIG. 9, row 3). Cells co-transfected with Cys-PDGFRTM, exhibited a clear and striking increase in binding to the CD38 antigen (see FIG. 9, row 4). This demonstrates the functional formation of the disulfide bond and the accompanying presentation of the antigen-binding IgG moiety on the eukaryotic cell surface.

As expected, cells transfected with the Cys-PDGFRTM construct (alone or in combination with Cys-IgG) expressed myc, as demonstrated by FACS (see FIG. 9). Likewise, IgG was detected when Cys-IgG was expressed.

Transfection with Cys-IgG also led to surface expression and antigen binding activity. However, co-transfection with Cys-PDGFRTM led to a significant increase in IgG staining as well as in antigen binding activity.

With HKB11 suspension cells a generally stronger expression was observed in FACS. Co-transfection of Cys-PDGFRTM and Cys-IgG also led to an increase in antigen binding activity as compared to transfection with Cys-IgG alone.

### Example 5

### Comparison of Cys-IgG Variants

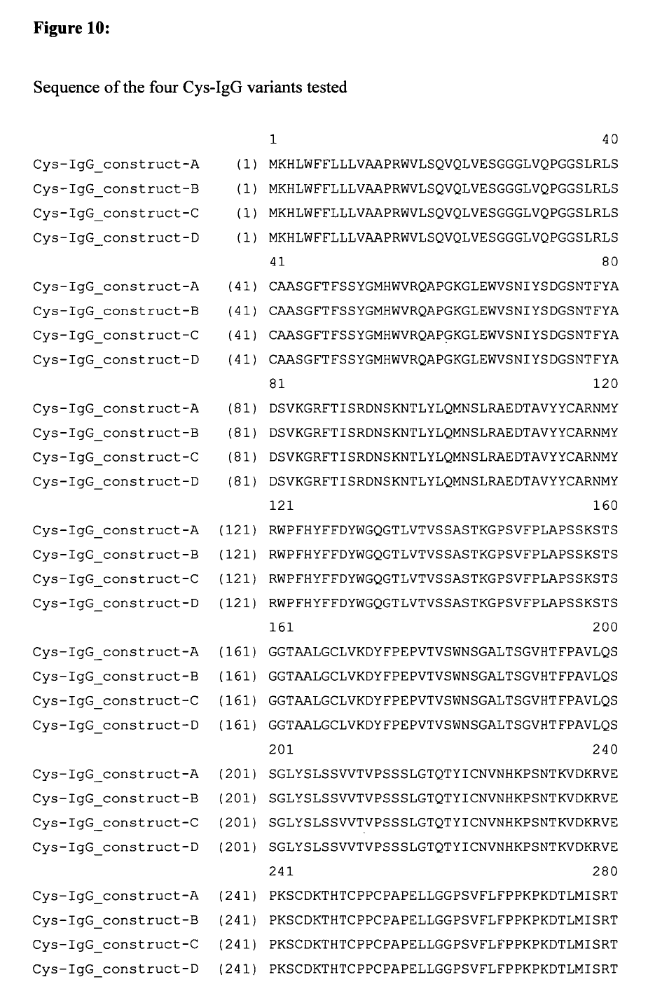

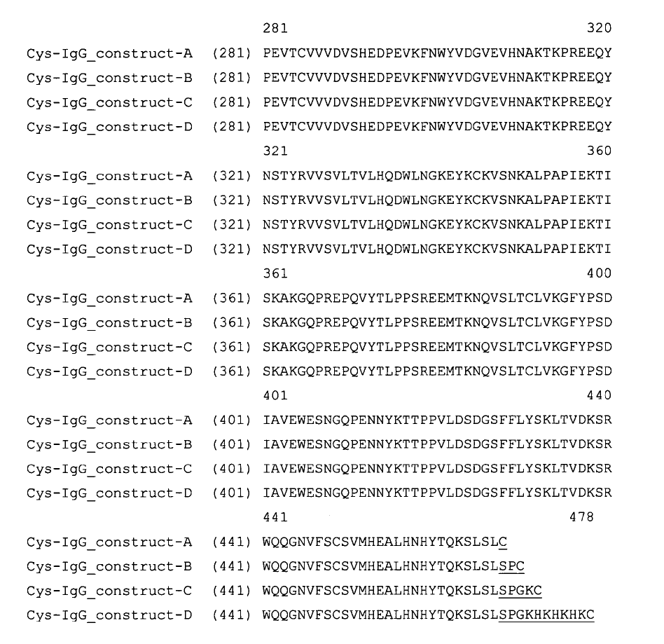

CHO cells were transiently transfected with four variants of Cys-IgG. The four variants differed in the C-termini (which comprise the reactive cysteine residues). The sequences of all four variants are shown in FIG. 10. One of the variants (construct C) represented the full-length IgG heavy chain with an additional cysteine residue at the very C-terminus. Constructs A and B are slightly shorter versions of IgG heavy chains, and construct D is a slightly extended version of an IgG heavy chain. All constructs comprise an additional cysteine residue at the very C-terminus. Construct A is identical to the Cys-IgG construct used in Example 4.

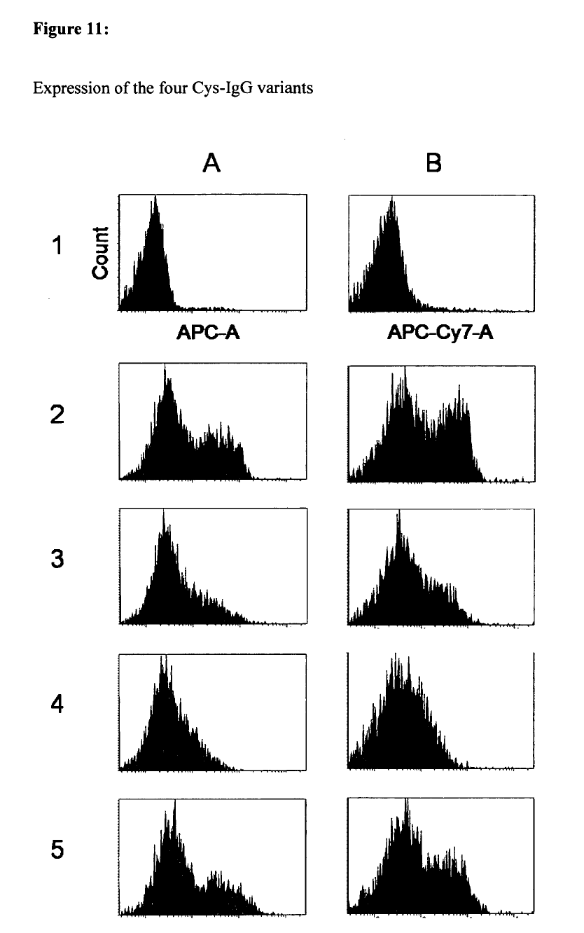

The experimental set up was the same as described above. Detection of IgG or antigen binding activity on the cell surface was performed as described above (columns A or B FIG. 11, respectively). Rows 1-5 (of FIG. 11) show the flow cytometric results of cells non-transfected or transfected with Cys-IgG constructs A-D, respectively.

All constructs led to expression of IgG at the eukaryotic cell surface and to respective antigen binding activity. Results are depicted in FIG. 11.

### Example 6

### Comparison of Cys-PDGFRTM Variants

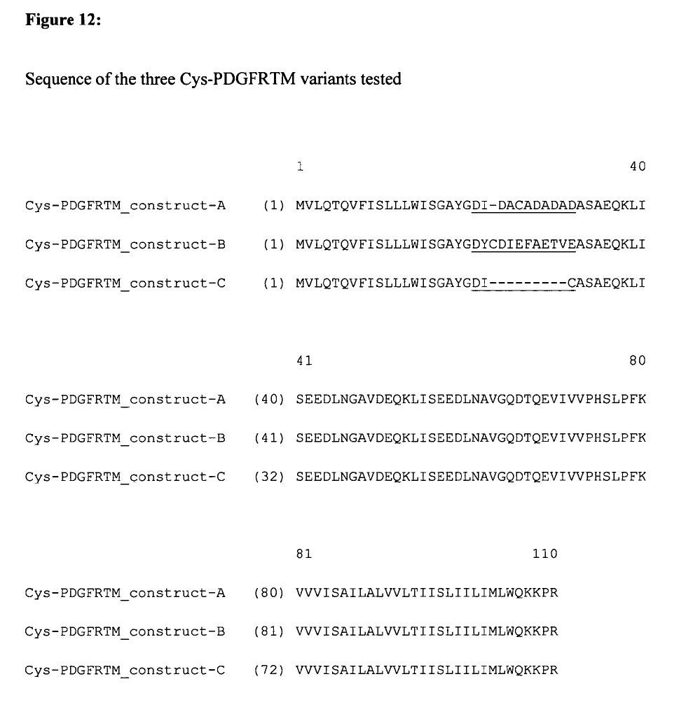

CHO cells were also transiently co-transfected with three variants of Cys-PDGFRTM differing in the neighboring amino acids of the reactive cysteine residue (underlined). The sequences of all four variants are shown in FIG. 12. Construct A depicted in FIG. 12 is identical to the Cys-PDGFRTM construct of Example 3.

Each of the variants of Cys-PDGFRTM was tested with each of the variants of Cys-IgG. Strikingly, all combinations led to the expression of IgG at the eukaryotic cell surface and to respective antigen binding activity. All Cys-PDGFRTM variants showed similar results.

### Example 7

### Analysis of Rebinding of Secreted IgG's

Purpose of this experiment was to confirm that in fact a disulfide bond is formed as purported in the present invention, and that the presentation of IgG molecules on the eukaryotic cell surface is not due to unspecific rebinding of secreted IgG.

One set of CHO cells was stably transfected to intracellularly express EGFP. Another set of CHO cells were transiently transfected with the Cys-IgG variant construct A (see Example 5). The two set of cells were combined after removal of transfection reagent (i.e. 6.5 hours after begin of transfection) and were co-cultured for 16 hours (see row 3 of FIG. 13). In control experiments non-transfected cells expressing EGFP or parental cells transfected with Cys-IgG were cultured separately under the same conditions with mock treatments (rows 1 and 2 of FIG. 13, respectively). The cell populations were then separately analyzed by FACS due to the expression or non-expression of EGFP.

Cell surface expression of Cys-IgG is depicted in column B of FIG. 13 (x-axis of diagrams). Column A of FIG. 13 shows the preparations without antibody staining. In these preparations signals could only be detected in the cells which permanently express EGFP (y-axis of diagrams), and only background signals could be detected with parental cells transfected with Cys-IgG.

Strikingly, only a small amount of Cys-IgG secreted from transfected cells was bound by EGFP expressing cells, as compared to the control cell populations. This convincingly demonstrates that the coupling of the genotype to the phenotype is completely retained. The same results were obtained with HKB11 suspension cells.

1. A vector comprising:
(a) a first polynucleotide capable of encoding a first (poly)peptide comprising at least one cysteine residue; and
(b) a second polynucleotide capable of encoding a second (poly)peptide which is a cell surface anchor comprising at least one cysteine residue,
wherein the vector is operable in a eukaryotic host cell to express said first (poly)peptide and said second (poly)peptide and to cause or allow the attachment of said first (poly)peptide to said second (poly)peptide by formation of a disulfide bond between said cysteine residue comprised within said first (poly)peptide and said cysteine residue comprised within said second (poly)peptide, wherein said first (poly)peptide is exhibited at the surface of a eukaryotic host cell.

2. The vector of claim 1, further comprising a signal sequence operably linked to the first polynucleotide.

3. The vector of claim 2, further comprising a signal sequence operably linked to the second polynucleotide.

4. The vector of claim 1, wherein said first (poly)peptide comprises a first chain of a binding molecule multi-chain (poly)peptide.

5. The vector of claim 4, further comprising:
(a1) a third polynucleotide capable of encoding a third (poly)peptide, wherein said third (poly)peptide comprises a second chain of the binding molecule multi-chain (poly)peptide.

6. The vector of claim 5, further comprising:
(a2) a fourth polynucleotide capable of encoding a fourth (poly)peptide, wherein said fourth (poly)peptide comprises a third chain of the binding molecule multi-chain (poly)peptide.

7. The vector of claim 6, further comprising:
(a3) a fifth polynucleotide capable of encoding a fifth (poly)peptide, wherein said fifth (poly)peptide comprises a fourth chain of the binding molecule multi-chain (poly)peptide.

8. The vector of claim 7, wherein said first, second, third, fourth and/or fifth polynucleotide are functionally linked.

9. The vector of claim 8, wherein the multi-chain (poly)peptide comprises a two-chain (poly)peptide.

10. The vector of claim 8, wherein the multi-chain (poly)peptide comprises a four-chain (poly)peptide, wherein the four-chain (poly)peptide is comprised of two first chains and two second chains.

11. The vector of claim 8, wherein the multi-chain (poly)peptide comprises a two-chain (poly)peptide selected from the group consisting of: an immunoglobulin, a Fab fragment, an extracellular domain of a T cell receptor, a MHC class I molecule, and a MHC class II molecule.

12. The vector of claim 11, wherein the multi-chain (poly)peptide comprises an immunoglobulin (Ig) or an Ig fragment.

13. The vector of claim 12, wherein the multi-chain (poly)peptide comprises an immunoglobulin selected from the group consisting of: IgA, IgD, IgE, IgG and IgM.

14. The vector of claim 13, wherein, on expression, said first, and/or second, and/or third, and/or fourth chain is/are attached via said disulfide bond(s) to the (poly)peptide which is a cell surface anchor.

15. The vector of claim 14, wherein the cell surface anchor comprises a cell surface (poly)peptide or protein of a eukaryotic cell.

16. The vector of claim 15, wherein the cell surface anchor comprises a portion of a cell surface (poly)peptide or protein of a eukaryotic cell that anchors to the cell surface of the eukaryotic host cell.

17. The vector of claim 16, wherein the cell surface anchor is selected from the group consisting of: a-agglutinin, the a-agglutinin component Agalp and Aga2p, FLO1, PDGFR, PRIMA, and mDAF.

18. The vector of claim 16, wherein the portion of a cell surface (poly)peptide or protein is a transmembrane domain of said cell surface (poly)peptide or protein.

19. The vector of claim 18, wherein said transmembrane domain of said cell surface (poly)peptide or protein is a transmembrane domain of PDGFR.

20. The vector of claim 18, wherein either said at least one cysteine residue comprised in said first (poly)peptide or said at least one cysteine residue comprised in said second (poly)peptide has been introduced artificially.

21. The vector of claim 20, wherein said at least one cysteine residue comprised in said first (poly)peptide has been introduced artificially.

22. The vector of claim 20, wherein said at least one cysteine residue comprised in said second (poly)peptide has been introduced artificially.

23. The vector of claim 22, wherein said at least one cysteine residue comprised in said first (poly)peptide and said at least one cysteine residue comprised in said second (poly)peptide have been introduced artificially.

24. The vector of claim 1, wherein the eukaryotic host cell is a mammalian cell.

25. The vector of claim 24, wherein said mammalian cell is a HEK293 cell, a HKB11 cell or a CHO cell.

26. A composition comprising:
(a) a first vector comprising a first polynucleotide capable of encoding a first (poly)peptide comprising at least one cysteine residue, wherein said first (poly)peptide comprises a first chain of a binding molecule multi-chain (poly)peptide; and
(b) a second vector comprising
ba) a second polynucleotide capable of encoding a second (poly)peptide comprising at least one cysteine residue which is a cell surface anchor; and,
bb) a third polynucleotide capable of encoding a third (poly)peptide, wherein said third (poly)peptide comprises a second chain of the binding molecule multi-chain (poly)peptide; and,
wherein the vectors are operable in a eukaryotic host cell to express said first (poly)peptide and said second (poly)peptide and to cause or allow the attachment of said first (poly)peptide to said second (poly)peptide by formation of a disulfide bond between said cysteine residue comprised within said first (poly)peptide and said cysteine residue comprised within said second (poly)peptide, wherein, said first and said third (poly)peptide is exhibited at the surface of a eukaryotic host cell.

27. A eukaryotic host cell comprising a vector as defined in claim 1.

28. The eukaryotic host cell of claim 27 which is a mammalian host cell.

29. The mammalian host cell of claim 28, wherein said mammalian cell is a HEK293 cell, a HKB11cell or a CHO cell.

30. A vector library comprising a plurality of vectors according to claim 1, wherein said plurality is derived from a heterogeneous population of said first, and second (poly)peptide.

31. A eukaryotic display library comprising a population of eukaryotic cells collectively displaying a heterogeneous population of at least 10² (poly)peptides encoded by the vectors according to claim 1.

32. A eukaryotic host cell library obtainable by transfecting the vector library of claim 30 into a plurality of host cells.

33. A method for displaying a (poly)peptide encoded by the vector(s) according to claim 1 on the surface of a eukaryotic host cell comprising the steps of:
(a) introducing into a eukaryotic host cell at least one vector as defined in claim 1; and
(b) culturing the host cells under conditions suitable for expression of the (poly)peptides comprised in said vector(s) or said composition.

34. The method of claim 33 wherein said host cell is a mammalian cell.

35. A method comprising the steps of:
(a) transfecting a population of eukaryotic host cells with at least one vector as defined in claim 1, such that substantially each cell comprises a vector encoding a diverse binding member;
(b) culturing the host cells under conditions suitable for expression and display on the cell surface of the binding member comprised in said vector,
wherein the attachment of the binding member to the (poly)peptide which is a cell surface anchor is achieved by formation of a disulfide bond;
(c) allowing for binding of at least one binding member displayed on the cell surface to its target,
thereby allowing for the formation of a specific binding member-target complex; and
(d) eluting under reducing conditions the cells displaying the at least one specific binding member of step (c).

36. The method of claim 35, further comprising the additional step of
(c1) carrying out after step (c): washing of the cells which have not bound specifically to the target.

37. The method of claim 36, further comprising the step of determining the nucleic acid sequence of the specific binding member.

38. A method comprising the steps of:
(a) transfecting a population of eukaryotic host cells with at least one vector as defined in claim 1, wherein said vector or said composition comprise(s) a polynucleotide capable of encoding a (poly)peptide comprising a binding member capable of binding to a target;
and a gene of interest functionally linked to the (poly)peptide which is a cell surface anchor and/or functionally linked to said binding member;
(b) culturing the host cells under conditions suitable for expression and display on the cell surface of the binding member comprised in said vector,
wherein the attachment of the binding member to the (poly)peptide which is a cell surface anchor is achieved by formation of a disulfide bond;
(c) allowing for binding of the binding member displayed on the cell surface to its target,
thereby allowing for the formation of a specific binding member-target complex; and
(d) eluting under reducing conditions the cells displaying the specific binding member of step (c).

39. The method of claim 38 further comprising the additional step of
(c1) carrying out after step (c): washing of the cells which have not bound specifically to the target.

40. The method of claim 38, wherein said gene of interest is selected from the group consisting of a therapeutic (poly)peptide, an industrial (poly)peptide, and (poly)peptides used in research.

41. The method of claim 35, wherein said host cell is a mammalian cell.

42. The method of claim 41, wherein said mammalian cell is a HEK293 cell, a HKB11 cell or a CHO cell.
